(19) United States
(12) Patent Application Publication
(10) Pub. No.: US 2026/0294824 A1
(43) Pub. Date: Oct. 1, 2026

(54) GLUCONIC ACID-COATED SELENIUM NANOPARTICLES AND MANUFACTURING METHOD THEREOF, AND PHARMACEUTICAL COMPOSITION COMPRISING THE SELENIUM NANOPARTICLE AND APPLICATIONS THEREOF

(71) Applicant: TRUE DIAMOND HEALTH CO., LTD., Taoyuan City (TW)

(72) Inventors: Pan-Chyr Yang, Taoyuan City (TW); S.-Ja TSENG, Taoyuan City (TW); Zi-Xian LIAO, Taoyuan City (TW); Cheng-Han WEN, Taoyuan City (TW); Lin LU, Taoyuan City (TW); Chun-Lun CHIU, Taoyuan City (TW)

(73) Assignee: TRUE DIAMOND HEALTH CO., LTD., Taoyuan City (TW)

(21) Appl. No.: 19/268,698

(22) Filed: Jul. 14, 2025

(30) Foreign Application Priority Data
Mar. 31, 2025 (TW) 114112302

(51) Int. Cl. A61K 9/51; A61K 33/04; A61P 35/00; B82Y 5/00

(52) U.S. Cl. CPC A61K 9/5123; A61K 9/5192; A61K 33/04; A61P 35/00; B82Y 5/00

(57) ABSTRACT

The present invention provides a gluconic acid-coated selenium nanoparticle (GA-SeNP), which comprises a selenium nanoparticle and gluconic acid, wherein the gluconic acid is coated on the selenium nanoparticle to form a core-shell structure. GA-SeNPs can repolarize M2 macrophages into M1 macrophages and induce apoptosis of cancer cells through the generation of reactive oxygen species, thereby serving as the medicine for cancer treatment. A manufacturing method thereof is also provided.

### CROSS-REFERENCE TO RELATED APPLICATION

Pursuant to 35 U.S.C. § 119 (a), this application claims the benefits of the priority to Taiwan Patent Application No. 114112302, filed on Mar. 31, 2025, which is incorporated by reference herein by its entirety.

### BACKGROUND OF THE INVENTION

### 1. Field of the Invention

The present invention relates to a gluconic acid-coated selenium nanoparticle which can be comprised in a pharmaceutical composition for macrophage repolarization, immune response promotion and cancer treatment. The present invention further relates to a manufacturing method of the gluconic acid-coated selenium nanoparticle.

### 2. Description of the Prior Arts

Macrophages are white blood cells derived from monocytes, and are phagocytic cells that are responsible for clearing invading pathogens and harmful endogenous materials. As macrophages can travel throughout the body and can be recruited to lesions, they show significant promise to be developed into an immune cell therapy.

Cancer cells may change normal cells' metabolic pathways to increase lactate production within the tumor microenvironment. It is known that lactate is not an ordinary metabolic waste, and lactate can reduce the number of M1 macrophages (which promote inflammation and remove pathogens), and increase the number of M2 macrophages (which inhibit inflammation and promote repair), thereby suppressing the immune response and promoting cancer cells growth.

The main obstacle in transforming preclinical macrophage-reprogramming studies into successful clinical therapies is the insufficient efficacy. Therefore, there is an urgent need to find a way to effectively increase the proportion of M1 macrophages that promote inflammation and pathogen removal.

### SUMMARY OF THE INVENTION

To solve the aforementioned problems, the present invention provides a gluconic acid-coated selenium nanoparticle (GA-SeNP), comprising a selenium nanoparticle and gluconic acid, wherein the gluconic acid is coated on the selenium nanoparticle.

According to the present invention, the gluconic acid-coated selenium nanoparticles can repolarize M2 macrophages to M1 macrophages (macrophages from an M2 phenotype to an M1 phenotype), so as to enhance the anticancer ability of the immune system. Further, the gluconic acid-coated selenium nanoparticles per se (not mediated by M1 macrophages) can promote reactive oxygen species (ROS) generation in cancer cells and promote cancer cell apoptosis to demonstrate dual anticancer efficacies. Finally, the gluconic acid-coated selenium nanoparticles demonstrate good drug tolerance.

In one embodiment, the selenium nanoparticle has an approximately spherical shape.

In one embodiment, the selenium nanoparticle has an average diameter of 20 nm to 250 nm, for example, 20 nm, 50 nm, 80 nm, 110 nm, 140 nm, 170 nm, 200 nm, 230 nm or 250 nm. Preferably, the selenium nanoparticle has an average diameter of 30 nm to 70 nm, 80 nm to 120 nm or 180 nm to 220 nm. More preferably, the selenium nanoparticle has an average diameter of 30 nm to 70 nm.

In one embodiment, the gluconic acid-coated selenium nanoparticle has an average diameter of 25 nm to 300 nm, for example, 25 nm, 50 nm, 75 nm, nm, 125 nm, 150 nm, 175 nm, 200 nm, 225 nm, 250 nm, 275 nm or 300 nm. Preferably, the selenium nanoparticle has an average diameter of 35 nm to 95 nm.

In one embodiment, the gluconic acid-coated selenium nanoparticle has a Zeta potential of −10 mV to −50 mV, for example, −10 mV, −20 mV, −30 mV, −mV or −50 mV. Preferably, the gluconic acid-coated selenium nanoparticle has a Zeta potential of −15 mV to −18 mV. More preferably, the gluconic acid-coated selenium nanoparticle has a Zeta potential of −16 mV to −17 mV.

The present invention further provides a pharmaceutical composition, comprising the gluconic acid-coated selenium nanoparticle and a pharmaceutically acceptable carrier.

In one embodiment, the gluconic acid-coated selenium nanoparticle is in a concentration of 0.01 μg/g to 0.2 μg/g. Preferably, the gluconic acid-coated selenium nanoparticle is in a concentration of 0.05 μg/g to 0.15 μg/g. More preferably, the gluconic acid-coated selenium nanoparticle is in a concentration of 0.08 μg/g to 0.12 μg/g.

In one embodiment, the gluconic acid-coated selenium nanoparticle is in a concentration of 0.01 μg/mL to 0.2 μg/mL. Preferably, the gluconic acid-coated selenium nanoparticle is in a concentration of 0.05 μg/mL to 0.15 μg/mL. More preferably, the gluconic acid-coated selenium nanoparticle is in a concentration of 0.08 μg/mL to 0.12 μg/mL.

In one embodiment, the pharmaceutically acceptable carrier comprises phosphate buffer saline (PBS).

In one embodiment, the pharmaceutical composition further comprises granulocyte-macrophage colony-stimulating factor (GM-CSF), lactate oxidase (LOX) or a combination thereof.

According to the present invention, the combination of the gluconic acid-coated selenium nanoparticles with GM-CSF and/or LOX can increase tumor necrosis factor-α (TNF-α) secretion and reduce interleukin-10 (IL-10) secretion, thereby enhancing immune responses and inhibiting cancer cells.

In one embodiment, the granulocyte-macrophage colony-stimulating factor is in a concentration of 10 ng/ml to 50 ng/ml, for example, 10 ng/mL, 20 ng/ml, 30 ng/mL, 40 ng/ml or 50 ng/mL. Preferably, the granulocyte-macrophage colony-stimulating factor is in a concentration of 22 ng/ml to 28 ng/mL. More preferably, the granulocyte-macrophage colony-stimulating factor is in a concentration of 24 ng/ml to 26 ng/ml.

In one embodiment, the granulocyte-macrophage colony-stimulating factor is in a concentration of 10 ng/g to 50 ng/g, for example, 10 ng/g, 20 ng/g, 30 ng/g, 40 ng/g or 50 ng/g. Preferably, the granulocyte-macrophage colony-stimulating factor is in a concentration of 22 ng/g to 28 ng/g. More preferably, the granulocyte-macrophage colony-stimulating factor is in a concentration of 24 ng/g to 26 ng/g.

In one embodiment, the lactate oxidase is in a concentration of 0.01 U/mL to 0.05 U/mL, for example, 0.01 U/mL, 0.02 U/mL, 0.03 U/mL, 0.04 U/mL or 0.05 U/mL. Preferably, the lactate oxidase is in a concentration of 0.015 U/mL to 0.035 U/mL. More preferably, the lactate oxidase is in a concentration of 0.022 U/mL to 0.028 U/mL.

In one embodiment, the lactate oxidase is in a concentration of 0.01 U/g to 0.05 U/g, for example, 0.01 U/g, 0.02 U/g, 0.03 U/g, 0.04 U/g or 0.05 U/g. Preferably, the lactate oxidase is in a concentration of 0.015 U/g to 0.035 U/g. More preferably, the lactate oxidase is in a concentration of 0.022 U/g to 0.028 U/g.

In one embodiment, the pharmaceutical composition is in an injection dosage form and can be used for injection for tumor therapy. Preferably, the pharmaceutical composition is in a dosage form of an injection for tumor therapy.

The present invention further provides a method for macrophage polarization, comprising contacting the pharmaceutical composition with a macrophage.

The present invention further provides a method for macrophage polarization, comprising providing an effective amount of the pharmaceutical composition. Preferably, the macrophage polarization comprises repolarization from an M2 macrophage to an M1 macrophage.

In one embodiment, the dose of the pharmaceutical composition for mice is 1 microgram (μg) to 50 μg per kilogram of body weight daily, for example, 1 μg, 5 μg, 10 μg, 15 μg, 20 μg, 25 μg, 30 μg, 40 μg or 50 μg. Preferably, the dose of the pharmaceutical composition for mice is 5 μg to 20 μg per kilogram of body weight daily. More preferably, the dose of the pharmaceutical composition for mice is 10 μg to 15 μg per kilogram of body weight daily.

Concerning the dose for humans, based on an adult with a body weight of 60 kg, the dose for mice/12.3=the dose for humans. Therefore, the dose of the pharmaceutical composition for humans is 0.08 μg to 4.1 μg per kilogram of body weight daily, for example, 0.08 μg, 0.1 μg, 0.5 μg, 1 μg, 1.5 μg, 2 μg, 2.5 μg, 3 μg, 3.5 μg, 4 μg or 4.1 μg. Preferably, the dose of the pharmaceutical composition for humans is 0.4 μg to 1.7 μg per kilogram of body weight daily.

More preferably, the dose of the pharmaceutical composition for humans is 0.8 μg to 1.3 μg per kilogram of body weight daily.

The present invention further provides a method for cancer treatment, comprising administering to a subject in need thereof an effective amount of the pharmaceutical composition.

In one embodiment, the cancer comprises melanoma.

In one embodiment, the cancer treatment comprises promoting reactive oxygen species (ROS) generation, promoting apoptosis of cancer cells, increasing tumor necrosis factor-α (TNF-α) secretion, reducing interleukin-10 secretion (IL-10) or a combination thereof. Preferably, the promoting reactive oxygen species generation is promoting reactive oxygen species generation in cancer cells.

In one embodiment, the treatment comprises amelioration and/or cure.

The present invention further provides a method for immune response promotion, comprising administering to a subject in need thereof an effective amount of the pharmaceutical composition. In the present invention, the pharmaceutical composition can be delivered to specific lesions (for example, the tumors) to increase the proportion of M1 macrophages at the lesions, thereby promoting immune responses. Preferably, the immune response promotion comprises promoting reactive oxygen species generation, inducing apoptosis at lesion, increasing tumor necrosis factor-α secretion, reducing interleukin-10 secretion or a combination thereof. More preferably, the promoting reactive oxygen species generation is promoting reactive oxygen species generation in cancer cells.

The present invention further provides a manufacturing method for the gluconic acid-coated selenium nanoparticle, comprising:
a mixing step: mixing sodium selenite (Na₂SeO₃), glucose, polyvinylpyrrolidone and water to obtain a mixture;
a heating step: heating the mixture to obtain a dark reddish-brown solution; and
a cooling step: lowering a temperature of the dark reddish-brown solution to obtain the gluconic acid-coated selenium nanoparticle.

According to the present invention, polyvinylpyrrolidone is a modifier for adjusting the size of the gluconic acid-coated selenium nanoparticles.

Without polyvinylpyrrolidone, the size of the gluconic acid-coated selenium nanoparticles will be too big to be absorbed.

In one embodiment, the mixing step comprises: dissolving sodium selenite in water to obtain a sodium selenite solution; glucose is added to the sodium selenite solution and then polyvinylpyrrolidone is further added thereto and mixed to obtain the mixture. Preferably, the mixing time to obtain the mixture is 15 minutes to 30 minutes. More preferably, the mixing time to obtain the mixture is 18 minutes to 22 minutes.

In one embodiment, in the mixing step, 0.5 g to 1.5 g of glucose needs to be mixed with 0.5 g sodium selenite. Preferably, 0.8 g to 1.2 g of glucose needs to be mixed with 0.5 g sodium selenite.

In one embodiment, in the mixing step, 0.5 mg to 2 mg of polyvinylpyrrolidone needs to be mixed with 0.5 g of sodium selenite. Preferably, 0.6 mg to 1.6 mg of polyvinylpyrrolidone needs to be mixed with 0.5 g of sodium selenite. More preferably, 0.8 mg to 1.2 mg of polyvinylpyrrolidone needs to be mixed with 0.5 g of sodium selenite.

In one embodiment, the temperature of the heating step is heating to 80° C. to 90° C. Preferably, the temperature of the heating step is heating to 83° C. to 87° C.

In one embodiment, the time of the heating step is 20 minutes to 40 minutes. Preferably, the time of the heating step is 27 minutes to 33 minutes.

In one embodiment, the cooling step is to reduce the temperature of the dark reddish-brown solution to room temperature. Preferably, the room temperature is 22° C. to 28° C.

To sum up, the gluconic acid-coated selenium nanoparticles (GA-SeNPs) of the present invention per se have anticancer efficacy. Further, GA-SeNPs can repolarize macrophages from an M2 macrophage to an M1 macrophage, and induce tumor cell apoptosis to further enhance the anticancer efficacy. Finally, the combination of the GA-SeNPs of the present invention with granulocyte-macrophage colony-stimulating factor and/or lactate oxidase can further enhance the repolarization proportion of macrophages from an M2 macrophage to an M1 macrophage to further enhance the anticancer efficacy.

### BRIEF DESCRIPTION OF THE DRAWINGS

FIG. 1 is the transmission electron microscope photo of Example 1.

FIG. 2 is the Zeta potential analysis diagram of Example 1.

FIG. 3 is the structural diagram of the gluconic acid-coated selenium nanoparticles.

FIG. 4 is the energy-dispersive X-ray spectroscopy analysis result of Example 2.

FIG. 5 is the Fourier-transform infrared spectroscopy analysis diagram of Example 2.

FIG. 6 is the X-ray diffraction analysis diagram of Example 2.

FIG. 7 is the cytotoxicity analysis bar chart of all groups of gluconic acid-coated selenium nanoparticles.

FIG. 8 is the biomarkers and nucleus staining photos for confirming macrophage repolarization results of all groups.

FIG. 9 is the quantitative bar chart of the macrophage repolarization results of all groups.

FIG. 10 and FIG. 11 are respectively the photos and the quantitative bar chart of cancer cell migration inhibition of all groups.

FIG. 12 shows the staining photos for confirming reactive oxygen species generation and apoptosis of all groups.

FIG. 13 is the bar chart of cancer cell growth inhibition results of all groups.

FIG. 14 is the quantitative bar chart of the macrophage polarization results of Example 1 in combination with different additives.

FIG. 15 is the quantitative bar chart of the secretion of tumor necrosis factor-α and interleukin-10 of Example 1 in combination with different additives.

FIG. 16 is the line chart of the body weight changes of mice of all groups.

FIG. 17 is the bar chart of the mouse spleen weights of all groups.

FIG. 18 to FIG. 20 are respectively the photos of mouse tumors, and bar charts of mouse tumor volumes and weights of all groups.

FIG. 21 is the apoptosis staining photos of mouse tumor sections of all groups.

### DETAILED DESCRIPTION OF THE PREFERRED EMBODIMENTS

The present invention is further explained through the following embodiments. A person having ordinary skill in the art can easily understand the advantages and efficacies achieved by the present invention. The present invention should not be limited to the contents of the embodiments. A person having ordinary skill in the art can make improvement or modifications without departing from the spirit and scope of the present invention to practice or apply the content of the present invention.

### 1. Gluconic Acid-Coated Selenium Nanoparticles (GA-SeNPs)

### (1) Example 1: GA-SeNPs Wherein the Selenium Nanoparticles Thereof Per Se (without Gluconic Acids) have a Diameter of 30 nm to 70 nm

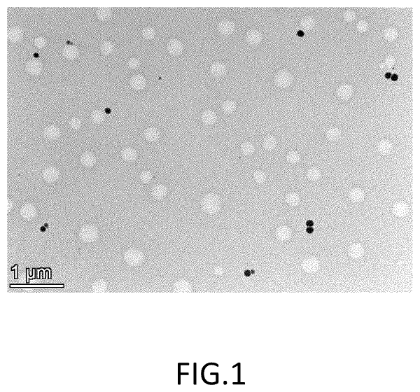

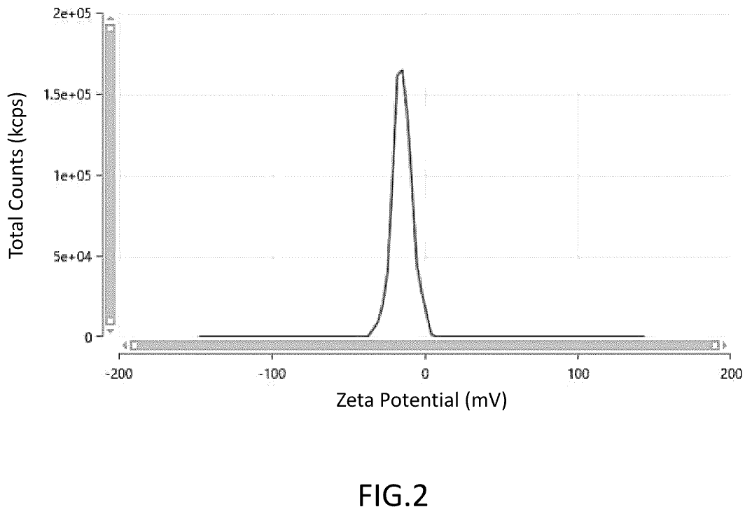

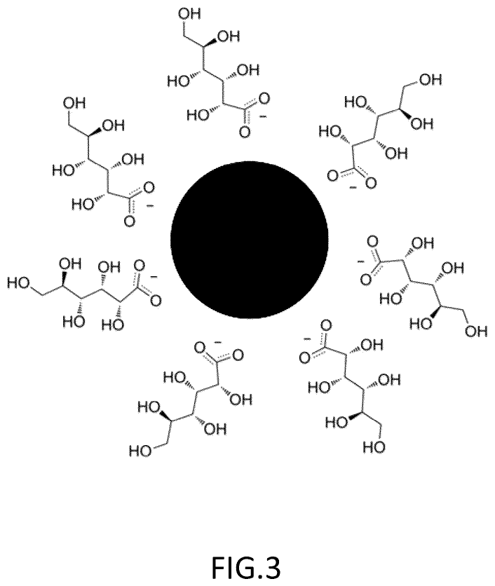

I. Steps: 0.5 g of sodium selenite (Na₂SeO₃) was dissolved in 5 mL of deionized water, and poured into a triangular conical flask with a capacity of 30 ml to obtain a reaction bottle. 1.1 g of glucose (Glu, C₆H₁₂O₆, as a reducing agent and a modifier) was gradually added into the reaction bottle. 0.7 mL of polyvinylpyrrolidone (PVP) aqueous solution in a concentration of 1 mg/ml was further added thereto and was stirred with a magnetic bar at room temperature (about 25° C.) for 20 minutes until a uniform mixture was obtained. The uniform mixture was placed on an electric hot plate set at 85° C. and stirred for 30 minutes to obtain a heated mixture in a color of dark reddish brown, and then the heated mixture was cooled to room temperature to obtain a GA-SeNPs suspension. Further, the GA-SeNPs suspension was centrifugated at 12,000 rpm for 20 minutes, and the supernatant thereof was removed to obtain colloidal GA-SeNPs.
II. Analysis:
A. Transmission electron microscope (TEM) analysis: The sample of 5 μL the GA-SeNPs suspension (not the colloidal GA-SeNPs) was spotted on a 200-mesh copper wire mesh, and heated on a heating plate set at 80° C. to fix the sample to the carbon film of the copper wire mesh. After the copper wire mesh with the sample was dried, the diameters of the selenium nanoparticles (without gluconic acids) were observed and measured through Spectra 300 FEG-S/TEM (purchased from Thermo Fisher). For clarification, gluconic acids, which were organic substances and unstable under high pressure, would be easily scattered and cannot be displayed during photographing.
B. Zeta potential analysis: The GA-SeNPs suspension (not the colloidal GA-SeNPs) was subjected to 2× dilution as a sample, and 1 ml was placed in a plastic sample tank equipped with a U-shaped gold electrode in Zetasizer Pro Blue (purchased from Malvern Panalytical) for measurement; wherein the solvent was deionized water, and the electrolyte was potassium chloride with a concentration of 0.001 mol/l.
C. Nanoparticle Tracking Analysis (NTA): The GA-SeNPs suspension (non-colloidal GA-SeNPs) was appropriately diluted with deionized water as a sample, and was injected into the flow cell of Nano Sight NS300 through 1 ml microinjector without generation of bubbles. After the laser intensity and camera sensitivity were adjusted, the image recording was carried out for 30 to 60 seconds for particle size and concentration analysis.
III. Results: First, the TEM analysis results are shown in FIG. 1; wherein the light-colored particles are dried sodium hydroxide particles, and the dark-black particles are selenium nanoparticles per se (without gluconic acids). According to the scale bar, the selenium nanoparticles per se have a diameter of about 30 nm to 70 nm, and selenium nanoparticles per se are spherical. Second, the Zeta potential analysis results are shown in FIG. 2; wherein Example 1 has a Zeta potential of −16.25 mV. Third, the particle size analysis result of NTA shows the average diameter of the GA-SeNPs (comprising selenium nanoparticles and gluconic acids) in Example 1 is mainly 68 nanometers, and the GA-SeNPs with the average diameter of 68 nanometers is in a concentration of 4.0 particles/ml.
IV. Notes: The chemical reaction formula for preparing GA-SeNPs is: Na₂SeO₃+2C₆H₁₂O₆→□Na₂O+Se+2C₆H₁₂O₇; wherein (1) Na₂O will form NaOH in the aqueous solution, so the present invention does not require the addition of alkali solution; (2) Glucose reduces sodium selenite into selenium nanoparticles, and glucose becomes gluconic acids with a carboxyl group. The carboxyl group adheres to the surface of the selenium nanoparticles to form a core-shell structure of selenium nanoparticles coated with gluconic acids (carboxyl groups), as shown in FIG. 3, to fix the shape and uniformize the diameter of the selenium nanoparticles; (3) The surfaces of GA-SeNPs are negatively charged, which can avoid aggregation of GA-SeNPs, and the size of GA-SeNPs will not be affected by centrifugation; (4) The diameter of the selenium nanoparticles per se (without gluconic acids) in both the GA-SeNPs suspension and the colloidal GA-SeNPs is 30 nm to 70 nm; and (5) The present invention controls the size of selenium nanoparticles by adding polyvinylpyrrolidone.

### (2) Example 2: GA-SeNPs Wherein the Selenium Nanoparticles Thereof Per Se (without Gluconic Acids) have a Diameter of 80 nm to 120 nm

### I. Steps:

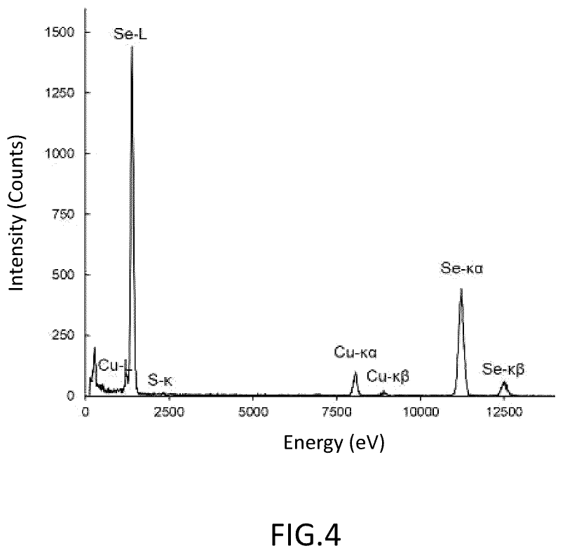

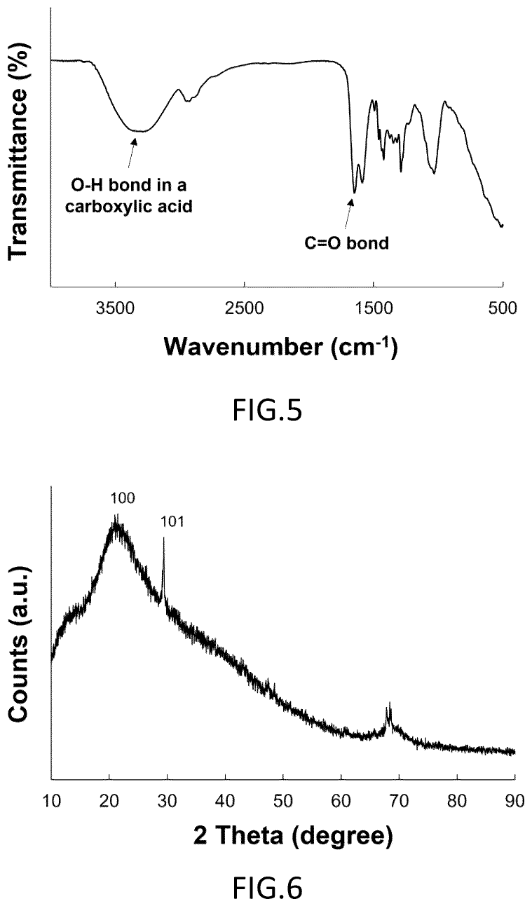

0.5 g of sodium selenite was dissolved in 5 mL of deionized water, and poured into a triangular conical flask with a capacity of 30 ml to obtain a reaction bottle. 1 g of glucose was gradually added into the reaction bottle. 1 mL of PVP aqueous solution in a concentration of 1 mg/mL was further added thereto and was stirred with a magnetic bar at room temperature (about 25° C.) for 20 minutes until a uniform mixture was obtained. The uniform mixture was placed on an electric hot plate set at 85° C. and stirred for 30 minutes to obtain a heated mixture in a color of dark reddish brown, and then the heated mixture was cooled to room temperature to obtain a GA-SeNPs suspension. Further, the GA-SeNPs suspension was centrifugated at 12,000 rpm for 20 minutes, and the supernatant thereof was removed to obtain colloidal GA-SeNPs.
II. Analysis:
A. The steps of both the TEM analysis and Zeta potential analysis of Example 2 were the same as those of Example 1.
B. Element analysis: The sample preparation process was the same as that of TEM analysis, and the sample was subjected to Energy-dispersive X-ray spectroscopy (EDS) analysis by Spectra 300 FEG-S/TEM (purchased from Thermo Fisher).
C. Fourier-transform infrared spectroscopy (FTIR) analysis: The GA-SeNPs suspension (not the colloidal GA-SeNPs) was vacuum-dried to obtain a solid sample, which was further ground into fine powders with a mortar and then placed on a ZnSe prism of FT/IR4600 (purchased from JASCO) for spectral measurement.
D. X-ray diffraction analysis (XRD): The GA-SeNPs suspension (not the colloidal GA-SeNPs) was freeze-dried for water removal to obtain a dark brown viscous sample. The dark brown viscous sample was placed on a silicon wafer of MiniFlex (purchased from Rigaku) for analysis, and had a thickness less than 50 microns.
III. Results: First, the selenium nanoparticles per se (without gluconic acids) of Example 2 have a diameter of 80 nm to 120 nm, and the GA-SeNPs of Example 2 have a Zeta potential of −16.25 mV, same as that of the GA-SeNPs of Example 1. Second, FIG. 4 demonstrates the EDS analysis result of Example 2, and confirms that the selenium ions of sodium selenite are indeed reduced to selenium (metalloid) nanoparticles. Further, the copper signal in FIG. 4 comes from the copper wire mesh for sample preparation. As there is no high-intensity signal other than selenium and copper, high-purity GA-SeNPs are obtained in Example 2. Third, FIG. 5 demonstrates the FTIR analysis result of Example 2, and confirms that the GA-SeNPs of Example 2 have hydroxyl groups (O—H bonds) and carbonyl groups (C═O bonds). That is, the GA-SeNPs indeed have gluconic acids. Fourth, FIG. 6 demonstrates the XRD analysis result of Example 2, and confirms that the selenium nanoparticles per se (without gluconic acids) of Example 2 have (100) and (101) crystal planes.
IV. Notes: In comparison with Example 1, the selenium nanoparticles per se (without gluconic acids) of Example 2 have greater diameter, which results from slightly reducing the amount of glucose and increasing the amount of PVP aqueous solution in the preparation.

### (3) Example 3: GA-SeNPs Wherein the Selenium Nanoparticles Thereof Per Se (without Gluconic Acids) have a Diameter of 180 nm to 220 nm

I. Steps: 0.5 g of sodium selenite was dissolved in 5 mL of deionized water, and poured into a triangular conical flask with a capacity of 30 ml to obtain a reaction bottle. 1 g of glucose was gradually added into the reaction bottle. 1.5 mL of PVP aqueous solution in a concentration of 1 mg/mL was further added thereto and was stirred with a magnetic bar at room temperature (about 25° C.) for 20 minutes until a uniform mixture was obtained. The uniform mixture was placed on an electric hot plate set at 85° C. and stirred for 30 minutes to obtain a heated mixture in a color of dark reddish brown, and then the heated mixture was cooled to room temperature to obtain a GA-SeNPs suspension. Further, the GA-SeNPs suspension was centrifugated at 10,000 rpm for 20 minutes, and the supernatant thereof was removed to obtain colloidal GA-SeNPs.
II. Analysis: The steps of the TEM analysis of Example 3 were the same as those of Example 1.
III. Results: The TEM analysis result of Example 3 demonstrated that the selenium nanoparticles per se (without gluconic acids) of Example 3 have a diameter of 180 nm to 220 nm.
IV. Notes: In comparison with Example 1, the selenium nanoparticles per se (without gluconic acids) of Example 3 have greater diameter, which results from slightly reducing the amount of glucose and greatly increasing the amount of PVP aqueous solution in the preparation. Further, as the GA-SeNPs of Example 3 were expected to be negatively charged, no additional Zeta potential analysis was performed.

### (4) Comparative Example 1: GA-SeNPs (Comprising Selenium Nanoparticles and Gluconic Acids) with an Average Diameter of 800 nm

I. Steps: 0.5 g of sodium selenite was dissolved in 5 mL of deionized water, and poured into a triangular conical flask with a capacity of 30 ml to obtain a reaction bottle. 1 g of glucose was gradually added into the reaction bottle and was stirred with a magnetic bar at room temperature (about 25° C.) for 20 minutes until a uniform mixture was obtained. The uniform mixture was placed on an electric hot plate set at 85° C. and stirred for 30 minutes to obtain a heated mixture in a color of dark reddish brown, and then the heated mixture was cooled to room temperature to obtain a GA-SeNPs suspension.
II. Particle size analysis by Dynamic light scattering (DLS): The GA-SeNPs suspension (not the colloidal GA-SeNPs) was subjected to 2× dilution as a sample, and 1 ml of the diluted GA-SeNPs suspension was placed in a transparent plastic sample tank in Zetasizer Pro Blue (purchased from Malvern Panalytical) for measurement.
III. Results: The particle size analysis by DLS demonstrates that the GA-SeNPs (comprising selenium nanoparticles and gluconic acids) of Comparative example 1 have an average diameter of 800 nm.
IV. Notes: In comparison with the preparation of Example 1, no PVP aqueous solution was added in that of Comparative example 1, which results in that the particle size of GA-SeNPs of Comparative example 1 became too big to effectively deliver the selenium nanoparticles thereof into target cells. Therefore, no TEM or other analysis was performed for the selenium nanoparticles.

### 2. In Vitro Culture of M2 Macrophages:

Cell culture: The mouse RAW 264.7 cells (ATCC TIB-71), representing M0 macrophages, were cultured with a complete culture medium 21 in a constant environment of 37° C. incubator with 5% CO₂ for 2 to 3 days until approximately confluent, and then cultured with a differentiation medium for 24 hours to obtain differentiated macrophages. The differentiated macrophages mainly comprise M2 macrophages and hereinafter were referred to as “differentiated M2 macrophages” for use in subsequent experiments. That is, the differentiated M2 macrophages were obtained after the above differentiation process.

The complete culture medium comprises: (1) Dulbecco's modified Eagle medium (DMEM); (2) 10 volume percent fetal bovine serum; (3) Penicillin in a concentration of 100 U/mL; (4) Streptomycin in a concentration of 100 μg/mL; and (5) Sodium bicarbonate in a concentration of 3.7 g/L. The differentiation medium comprises DMEM and interleukin-4 (IL-4) in a concentration of 20 ng/mL.

### 3. The B16-F10 Murine Melanoma Cell Expressing Green Fluorescent Protein (GFP) (Abbreviated as “B16-F10-GFP Cell”)

The B16-F10 murine melanoma cells (ATCC CRL-6475) (abbreviated as “B16-F10 cells”) were seeded in a 12-well plate at a density of 1.4×10⁵ cells per well, and cultured in DMEM medium for 24 hours for adhesion. The lentivirus carrying GFP expression gene was purchased from National RNAi Core Facility at Academia Sinica (Taiwan). The B16-F10 cells were infected by said lentivirus according to the manual instruction for delivering the GFP expression gene into the B16-F10 cell, and cultured with DMEM medium comprising 10 volume percent fetal bovine serum, 100 U/mL penicillin, 100 μg/mL streptomycin and 2 μg/mL puromycin for 7 days for screening to obtain B16-F10-GFP cells for use in subsequent experiments.

### 4. Experiment 1: Cytotoxicity Analysis for GA-SeNPs

(1) This experiment intended to understand the cytotoxicity of GA-SeNPs of Example 1 to Example 3 (which have different diameters of the selenium nanoparticles) and the cytotoxicity of GA-SeNPs at different concentrations, and comprised the following groups:
I. The-Control groups: The test solutions for all Control groups were the same, which did not comprise GA-SeNPs (concentration: 0 μg/mL), and comprised DMEM medium (pH=7.4) added with 1M L-lactic acid until the pH of DMEM medium reached 6.7. Further, there were two kinds of cells for cytotoxicity analysis: differentiated M2 macrophages and B16-F10 cells, and 6 test solutions (2 kinds of cells×Examples 1 to 3) were prepared for the Control groups.
II. The-Experimental groups: There were 60 Experimental groups in total resulting from the combination of four factors as follows:
Factor 1: cell type. 2 kinds of cells were tested: differentiated M2 macrophages and B16-F10 cells.
Factor 2: the pH value of medium. 2 pH values of medium were tested: 6.7 and 7.4; wherein the medium comprised DMEM.
Factor 3: the diameter of the selenium nanoparticles. 3 diameters of the selenium nanoparticles of Examples 1 to 3 were tested.
Factor 4: the GA-SeNPs concentrations. 5 GA-SeNPs concentrations were tested: 0.01 μg/mL, 0.02 μg/mL, 0.05 μg/mL, 0.1 μg/mL and 0.2 μg/mL.

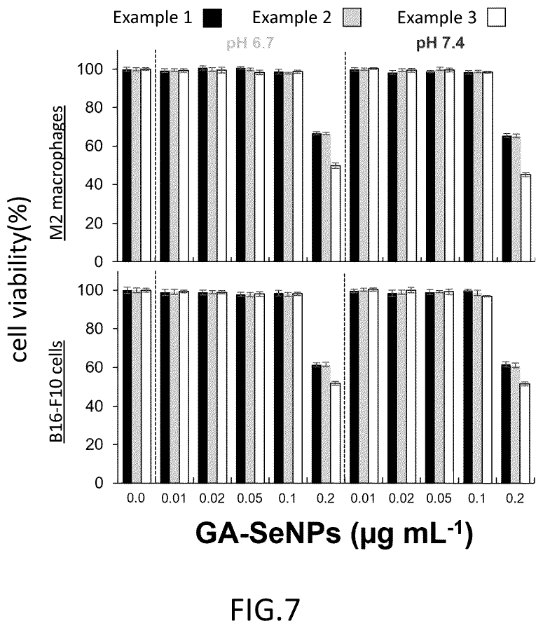

From above, 60 (=2×2×3×5) test solutions were prepared for the Experimental groups. For clarification, even there were Experimental groups set for the mediums with a pH value of 6.7 and 7.4, there were only one Control group set for pH 6.7, and no Control group was set for pH 7.4 in this experiment.
(2) Repolarization steps: The differentiated M2 macrophages and B16-F10 cells were respectively seeded in 96-well plates at a density of 1×10⁴ cells per well and cultured in DMEM overnight for cell adhesion, and then the media were replaced with the test solution of each group and cultured for 24 hours. Finally, the kit of the CellTiter 96 AQueous One Solution Cell Proliferation Assay kit (purchased from Promega) was used to obtain cell survival rates for cytotoxicity analysis.
(3) Results: According to FIG. 7, all groups with 0.2 μg/mL GA-SeNPs demonstrated cytotoxicity for both differentiated M2 macrophages and B16-F10 cells, while the factors of medium pH values and diameters of selenium nanoparticles did not demonstrate cytotoxicity. On the other hand, all groups with 0.01 μg/mL, 0.02 μg/mL, 0.05 μg/mL and 0.1 μg/mL GA-SeNPs demonstrated no cytotoxicity for both differentiated M2 macrophages and B16-F10 cells.

### 5. Experiment 2: Macrophage Repolarization Efficacy Analysis for GA-SeNPs

(1) This experiment intended to understand whether the GA-SeNPs can repolarize the differentiated M2 macrophages into M1 macrophages, and comprised the following groups:
I. The Control groups: the test solutions for all Control groups did not comprise GA-SeNPs (concentration: 0 μg/mL), and the test solutions for the Control groups comprised DMEM with a pH value of 6.7 and 7.4, respectively.
II. The Experimental groups: There were 24 Experimental groups in total resulting from the combination of three factors as follows:
Factor 1: the pH value of medium. 2 pH values of medium were tested: 6.7 and 7.4; wherein the medium comprised DMEM.
Factor 2: the diameter of the selenium nanoparticles. 3 diameters of the selenium nanoparticles of Examples 1 to 3 were tested.
Factor 3: the GA-SeNPs concentrations. 4 GA-SeNPs concentrations were tested: 0.01 μg/mL, 0.02 μg/mL, 0.05 μg/mL and 0.1 μg/mL.

From above, 24 (=2×3×4) test solutions were prepared for the Experimental groups.
(2) Repolarization steps: The differentiated M2 macrophages were seeded in 24-well plates at a density of 7×10⁴ cells per well and cultured in DMEM overnight for cell adhesion. The media were further replaced with the test solution of each group and the cells were cultured for 72 hours; wherein all the test solutions were renewed every 24 hours.
(3) Qualitative Analysis:
I. Biomarkers and nuclear staining

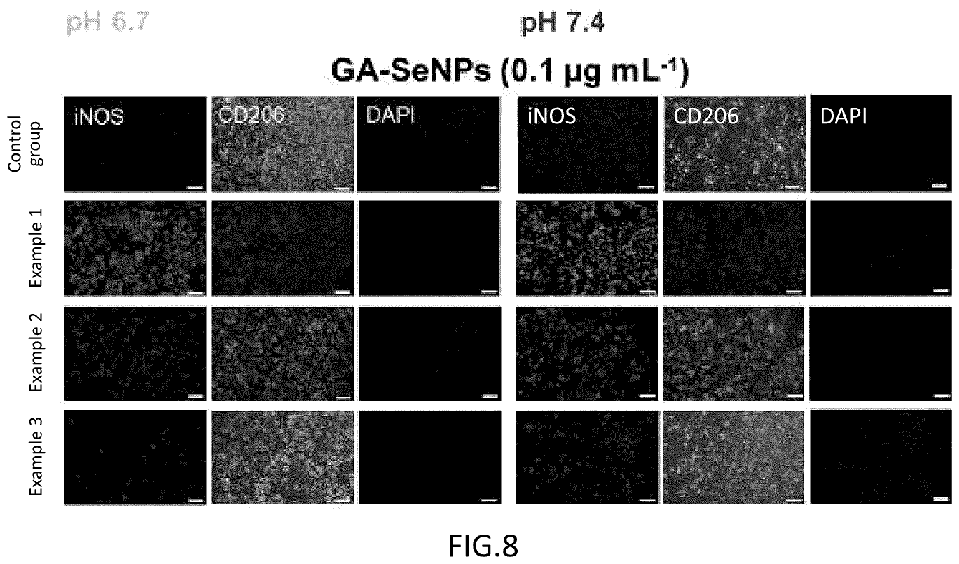

The biomarkers of M2 macrophages comprised CD206, and no iNOS; whereas that of M1 macrophages comprised iNOS, and no CD206. In this analysis, the macrophages after the repolarization steps were fixed with 4% paraformaldehyde (PFA), and immunostaining was carried out with the anti-iNOS antibody (purchased from Abcam) and anti-CD206 antibody (purchased from R&D system). Further, rabbit anti-goat IgG antibody-FITC (purchased from Genetex, green fluorescence) was used to detect CD206 for signal amplification, and goat anti-rabbit IgG (H+L)-TAMRA (purchased from Leadgene, Taiwan, red fluorescence) was used to detect iNOS for signal amplification. This analysis also performed DAPI nuclear staining. Finally, the staining results were observed with a confocal microscope.
II. Results: The photos of the staining results of all groups corresponding to 0.1 μg/mL GA-SeNPs are taken, and demonstrated in FIG. 8, wherein the scale bar therein is 200 microns. According to FIG. 8, in the Control groups, the staining signals of CD206 in the Control groups are significantly greater than those of iNOS, which indicates that the test solutions in the Control groups cannot repolarize the differentiated M2 macrophages into M1 macrophages (iNOS⁺CD206⁻), as the differentiated M2 macrophages after the repolarization steps still demonstrate the phenotype of M2 macrophages (iNOS-CD206+). In comparison, in the Experimental group of Example 1, the staining signals of iNOS are significantly greater than those of CD206, which indicates that the test solution with Example 1 in a concentration of 0.1 μg/mL can effectively repolarize the differentiated M2 macrophages into M1 macrophages (iNOS⁺CD206⁻) to demonstrate the phenotype of M1 macrophages.

### (4) Quantitative Analysis:

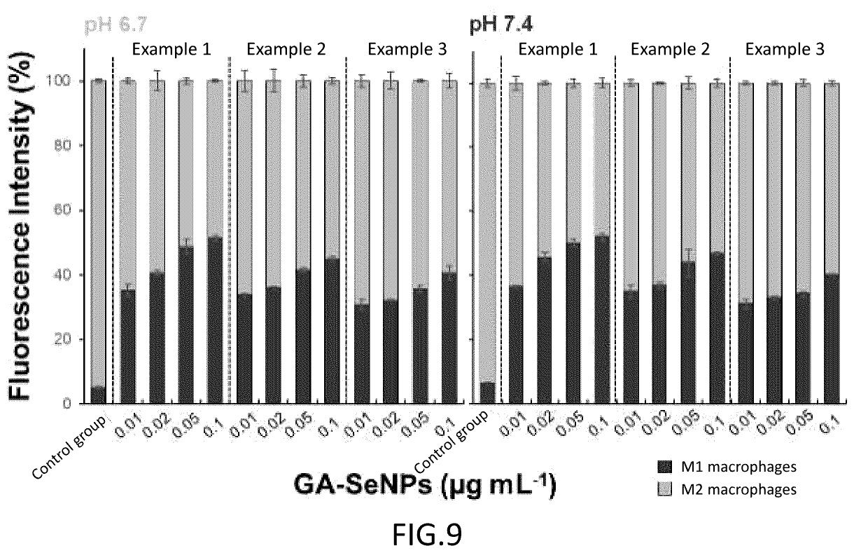

I. Analysis steps: This analysis used flow cytometry (purchased from Beckman Coulter) to quantitatively analyze the cells after immunostaining; wherein the cells were appropriately gated by forward and side scatter, and 10,000 events were collected for cell counting for each group.
II. Results: According to FIG. 9, the test solutions of the Control groups did not comprise GA-SeNPs, and in the repolarization results of the Control groups, the proportion of M1 macrophages are extremely low, and almost all cells are still M2 macrophages. In comparison, in the Experimental groups, a higher concentration of GA-SeNPs results in a higher proportion of M1 macrophages after the repolarization steps. Further, a smaller diameter of the selenium nanoparticles of GA-SeNPs results in a higher proportion of M1 macrophages after the repolarization steps. Therefore, GA-SeNPs can repolarize M2 macrophages into M1 macrophages.

### 6. Experiment 3: Anticancer Efficacy Analysis for GA-SeNPs

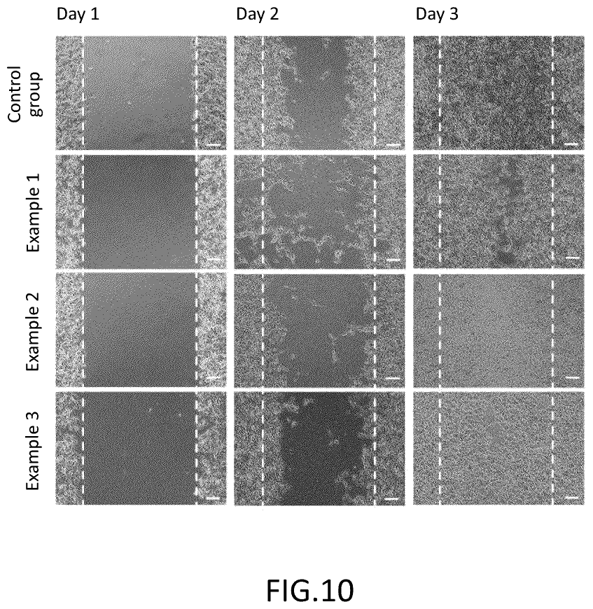

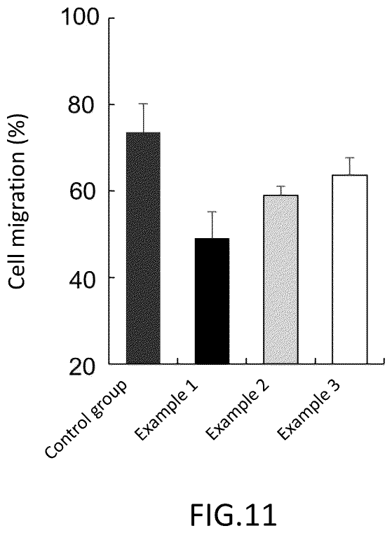

(1) This experiment intended to understand whether the GA-SeNPs can inhibit the cell migration of B16-F10 cells and can be used in anticancer therapy, and comprised the following groups:
I. The-Control groups: The test solutions for all Control groups did not comprise GA-SeNPs (concentration: 0 g/mL), and the test solutions for the Control group comprised DMEM with a pH value of 7.4.
II. The-Experimental groups: The test solutions for all Experimental groups comprised DMEM with a pH value of 7.4 and GA-SeNPs in a concentration of 0.1 μg/mL. Further, there were 3 Experimental groups in total; wherein the test solutions thereof comprised 3 kinds of diameters of the selenium nanoparticles: 3 diameters of the selenium nanoparticles of Examples to 3 were tested.
(2) Repolarization steps: The B16-F10 cells were seeded in 24-well plates at a density of 1×10⁶ cells per well and cultured in DMEM overnight for cell adhesion. A vertical scratch was then made across the center of each well by a pipette tip to create a cell-free zone, and the wells were washed twice with phosphate buffer saline (PBS) to remove any non-adherent cells. The test solutions were added, and all cells were cultured for 72 hours; wherein all the test solutions were renewed every 24 hours.
(3) Analysis: The migration of B16-F10 cells were observed with a microscope every day.
(4) Results: The scale bars in FIG. 10 and FIG. 11 are 200 microns. According to FIG. 10 and FIG. 11, a smaller diameter of selenium nanoparticles of GA-SeNPs results in a higher migration inhibition effect for B16-F10 cells, and GA-SeNPs indeed have anticancer efficacy.

### 7. Experiment 4: Anticancer Mechanism Analysis for GA-SeNPs

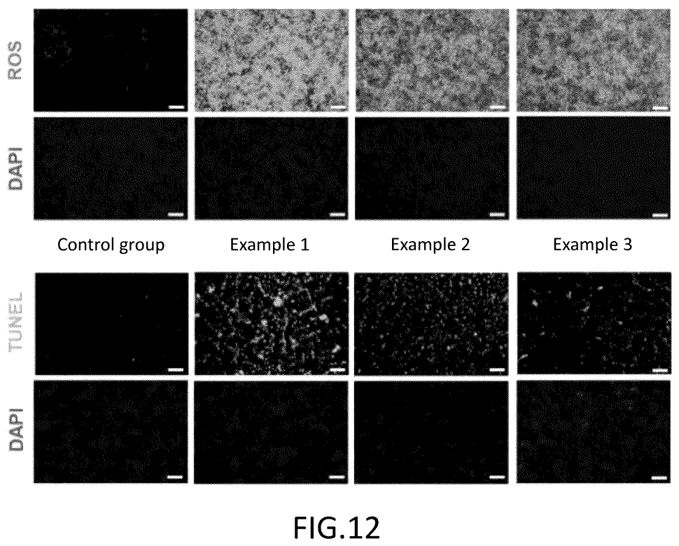

(1) This experiment intended to understand whether the GA-SeNPs can A. promote reactive oxygen species (ROS) generation, and B. promote apoptosis, and can be used in anticancer therapy, and comprised the following groups:
I. The-Control groups: the test solution for all Control groups did not comprise GA-SeNPs (concentration: 0 μg/mL), and the test solutions for the Control group comprised DMEM with a pH value of 7.4.
II. The Experimental groups: the test solutions for all Experimental groups comprised DMEM with a pH value of 7.4 and GA-SeNPs in a concentration of 0.1 μg/mL. Further, there were 3 Experimental groups in total; wherein the test solutions thereof comprised 3 kinds of diameters of the selenium nanoparticles: 3 diameters of the selenium nanoparticles of Examples 1 to 3 were tested.
(2) Repolarization steps: The B16-F10 cells were seeded in 24-well plates at a density of 1×10⁶ cells per well and cultured in DMEM overnight for cell adhesion. The media were further replaced with the test solution of each group and the cells were cultured for 72 hours; wherein all the test solutions were renewed every 24 hours.
(3) Analysis:
I. ROS analysis: CellROX green reagent can be oxidized by ROS and then combined with DNA to emit green fluorescence, and a stronger green fluorescence indicates a higher amount of ROS. This analysis used CellROX green reagent (purchased from Invitrogen) according to the manual instructions. CellROX green reagent was added to the test solutions 12 hours before analysis. DAPI nuclear staining was carried out for verification. Finally, the staining results were observed with a confocal microscope.
II. Apoptosis analysis: This analysis used the kit of Click-iT Plus TUNEL Assay Kits for In Situ Apoptosis Detection (purchased from Invitrogen) according to the manual instructions. DAPI nuclear staining was carried out for verification. Finally, the staining results were observed with a confocal microscope.
(4) Results: The scale bar in FIG. 12 is 200 microns. According to FIG. 12, the ROS and TUNEL staining signals of Example 1 are higher than those of other groups, which indicates that a smaller diameter of the selenium nanoparticles of GA-SeNPs results in a better efficacy to promote ROS generation and cell apoptosis. That is, a smaller diameter of the selenium nanoparticles of GA-SeNPs results in a better anticancer efficacy.

### 8. Experiment 5: Anticancer Efficacy Analysis for the Combination of GA-SeNPs and M2 Macrophages

(1) This experiment intended to understand the anticancer efficacy of the combination of GA-SeNPs with different numbers of M2 macrophages, and comprised the following groups:
I. The Control groups: the test solution for all Control groups did not comprise GA-SeNPs (concentration: 0 μg/mL), and the test solutions for the Control group comprised DMEM with a pH value of 7.4.
II. The Experimental groups: the test solutions for all Experimental groups comprised DMEM with a pH value of 7.4 and GA-SeNPs in a concentration of 0.1 μg/mL. Further, there were 12 Experimental groups in total resulting from the combination of two factors as follows:
Factor 1: numbers of M2 macrophages as shown in Table 1.
Factor 2: the diameter of the selenium nanoparticles. 3 diameters of the selenium nanoparticles of Examples 1 to 3 were tested.
From above, 12 (=4×3) test solutions were prepared for the Experimental groups.
(2) Repolarization steps: The cells were seeded in 24-well plates and cultured in DMEM overnight for cell adhesion. The media were further replaced with the test solution of each group and the cells were cultured for 72 hours; wherein all the test solutions were renewed every 24 hours. Further, the cells comprised: A. B16-F10-GFP cells: the seeded cell density thereof in all Control groups and Experimental groups was 1×10⁴ cells per well; and B. differentiated M2 macrophages: the details were shown in Table 1.

TABLE 1
the cell numbers of the differentiated M2 macrophages per well

| | Cell numbers of the differentiated M2 macrophages per |
|---|---|
| Groups | well:Cell numbers of the B16-F10-GFP cells per well |
| 0-fold group | 0:1 |
| 1-fold group | 1:1 |
| 3-fold group | 3:1 |
| 5-fold group | 5:1 |

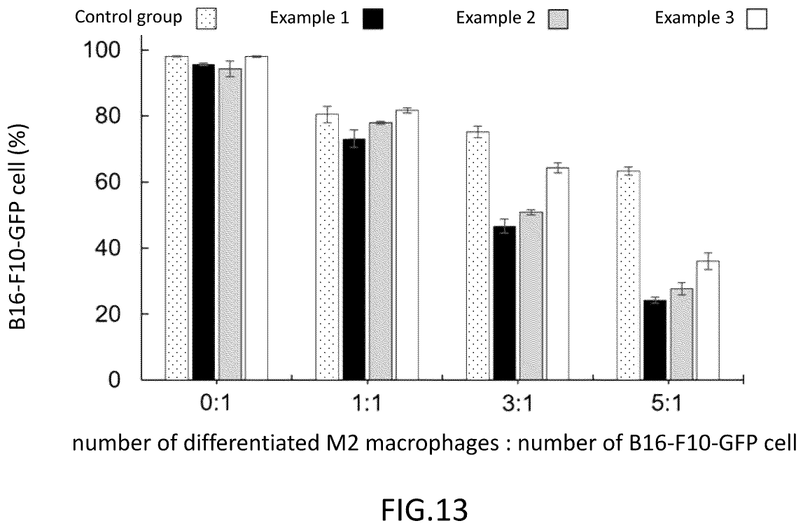

(3) Analysis: The cells in each group were fixed with 4% paraformaldehyde (PFA) solution and subjected to DAPI nuclear staining. The staining results were observed with a confocal microscope. Further, as B16-F10-GFP cells would emit green fluorescence, the number of B16-F10-GFP cells was calculated by flow cytometry. The percentage of B16-F10-GFP cells after the repolarization steps in each group was calculated based on the corresponding 0-fold group.
(4) Results: According to FIG. 13, first, in all Control groups, although the test solutions thereof comprised no GA-SeNPs, there were differentiated M2 macrophages in the 1-fold, 3-fold, and 5-fold groups. Therefore, the number of B16-F10-GFP cells decreased slightly. Second, in all 1-fold, 3-fold, and 5-fold groups of the Experimental groups, all groups with Example 1 have the lowest percentages of B16-F10-GFP cells and demonstrated the best anticancer efficacy in comparison with those with Examples 2 and 3. Third, in all groups that with Examples 1 to 3, the 3-fold and 5-fold groups demonstrated better anticancer efficacy than the 0-fold and 1-fold groups, which indicated that when the differentiated M2 macrophages have a number that was 3 times or more than that of B16-F10-GFP cells, their combination with GA-SeNPs can significantly enhance anticancer efficacy, as there would be more differentiated M2 macrophages to be repolarized into M1 macrophages by GA-SeNPs to significantly enhance anticancer efficacy.

### 9. Experiment 6: M2 Macrophages Repolarization Efficiency Analysis for GA-SeNPs

(1) This experiment intended to understand whether the combinations of GA-SeNPs with granulocyte-macrophage colony-stimulating factor (GM-CSF) and/or lactate oxidase (LOX) can A. increase the repolarization proportion of macrophages from an M2 macrophage to an M1 macrophage; and B. increase inflammatory cytokine secretion to enhance anticancer efficacy, and comprised the following groups:
I. The-Control groups: the test solutions for all Control groups did not comprise GA-SeNPs, GM-CSF and LOX, and the test solutions for the Control groups comprised DMEM with a pH value of 6.7 and 7.4, respectively.
II. The Experimental groups: the test solutions for all Experimental groups comprised 0.1 μg/mL GA-SeNPs of Example 1, and there were 8 Experimental groups in total resulting from the combination of three factors as follows:
Factor 1: the pH value of medium. 2 pH values of medium were tested: 6.7 and 7.4; wherein the medium comprised DMEM.
Factor 2: the addition of GM-CSF in a concentration of 25 ng/ml: yes or no.
Factor 3: the addition of LOX in a concentration of 25 ng/ml: yes or no.

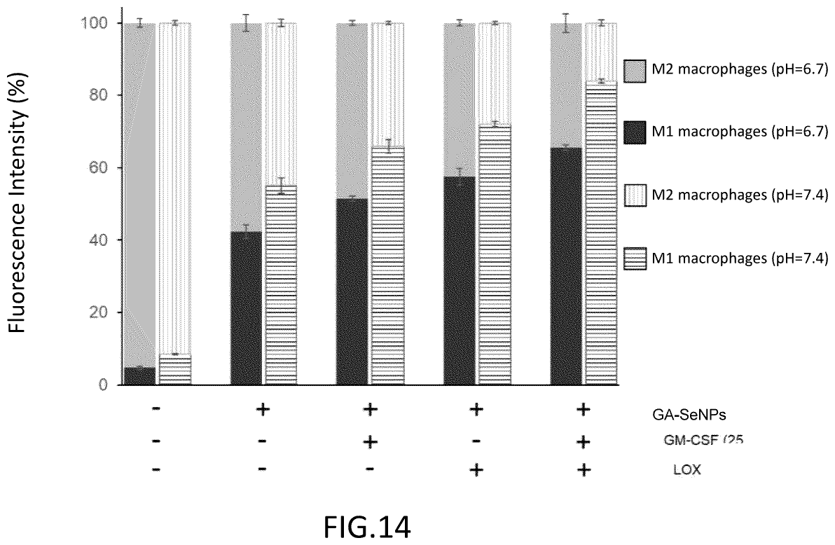

From above, 8 (=2×2×2) test solutions were prepared for the Experimental groups.
(2) Repolarization steps: The differentiated M2 macrophages were seeded in 24-well plates at a density of 7×10⁴ cells per well and cultured in DMEM overnight for cell adhesion. The media were further replaced with the test solution of each group and the cells were cultured for 72 hours; wherein all the test solutions were renewed every 24 hours.
(3) Analysis:
I. Biomarkers and nuclear staining: the steps were the same as those of Experiment 2. Briefly, all groups were subjected to biomarkers and nuclear staining to identify M1 macrophages (iNOS⁺CD206″) and M2 macrophages (iNOS-CD206+), and the flow cytometry (purchased from Beckman Coulter) was used to quantitatively analyze the cells after immunostaining; wherein the cells were appropriately gated by forward and side scatter, and 10,000 events were collected for cell counting for each group.
II. Enzyme-linked immunosorbent assay (ELISA): A. The detection kit of Mouse TNF-α ELISA Kit (purchased from BioLegend, Hsinchu) was used to measure the concentration of tumor necrosis factor-α (TNF-α) in each group; and B. The detection kit of Mouse IL-10 ELISA Kit (purchased from BioLegend, Hsinchu) was used to measure the concentration of interleukin-10 (IL-10).
(4) Results: According to FIG. 14, GA-SeNPs per se can repolarize macrophages from an M2 macrophage to an M1 macrophage, and their combination with GM-CSF or LOX can respectively promote the repolarization efficacy of GA-SeNPs to increase the proportion of repolarization macrophages from an M2 macrophage to an M1 macrophage. Further, the combination of GA-SeNPs and LOX increased more repolarization proportion than the combination of GA-SeNPs and GM-CSF, and the combination of GA-SeNPs, GM-CSF and LOX demonstrated the highest repolarization proportion. Besides, all groups using DMEM with a pH value of 7.4 had a higher repolarization proportion than those using DMEM with a pH value of 6.7, which indicated that a neutral microenvironment would be more suitable for GA-SeNPs to demonstrate repolarization efficacy.

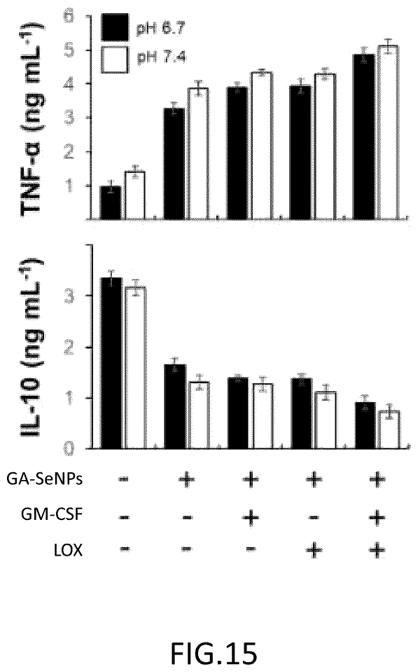

According to FIG. 15, concerning TNF-α, GA-SeNPs per se can increase TNF-α secretion, their combination with GM-CSF or LOX can further increase TNF-α secretion, respectively, and the combination of GA-SeNPs, GM-CSF and LOX demonstrated the highest TNF-α secretion. Concerning IL-10, GA-SeNPs per se can inhibit IL-10 secretion, their combination with GM-CSF or LOX can further inhibit IL-10 secretion, respectively, and the combination of GA-SeNPs, GM-CSF and LOX demonstrated the lowest IL-10 secretion.

### 10. Experiment 7: Animal Experiments

(1) This experiment intended to understand whether GA-SeNPs can inhibit melanoma xenograft tumors, and comprised the groups as shown in Table 2.

TABLE 2
Medical treatment for all groups

| Groups | Medical treatment |
|---|---|
| PBS group | From day 0 to day 8, 100 μl of PBS was injected into the |
| | tumor once a day. |
| GA-SeNPs (6 | From day 0 to day 5, 100 μl of PBS solution comprising |
| injections) | 0.25 μg of GA-SeNPs was injected into the tumor once a |
| group | day. That is, the dose was 12.5 μg of the GA-SeNPs of |
| | Example 1 per kilogram of body weight per day. |
| GA-SeNPs (9 | From day 0 to day 8, 100 μl of PBS solution comprising |
| injections) | 0.25 μg of GA-SeNPs was injected into the tumor once a |
| group | day That is, the dose was 12.5 μg of the GA-SeNPs of |
| | Example 1 per kilogram of body weight per day. |

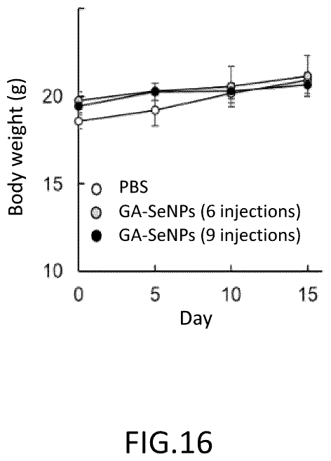

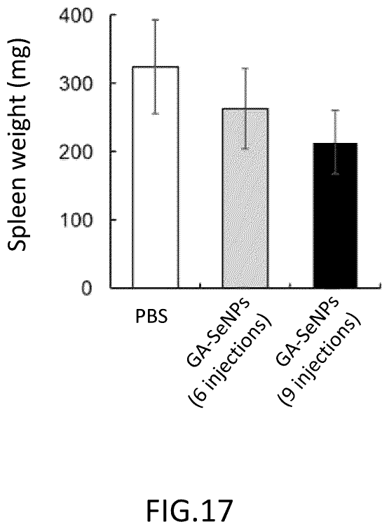

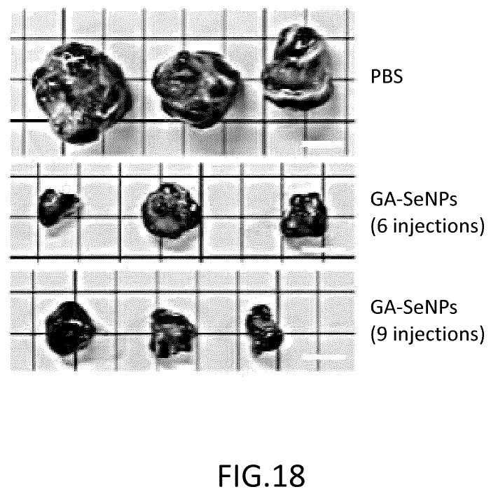

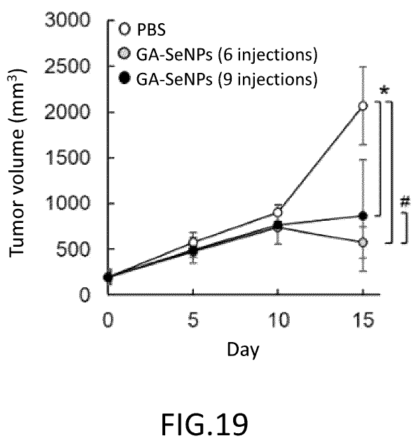

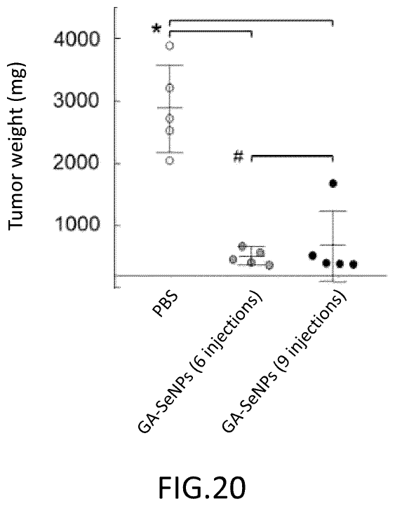

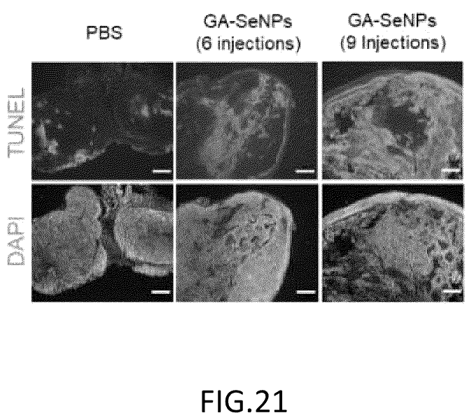

(2) Experiment treatments:
I. Experimental animals: Six-week-old male BALB/c mice, sourced from the National Laboratory Animal Center in Taiwan, were housed under controlled conditions with a 12-hour light/dark cycle. Up to five mice were housed per cage, with unrestricted access to food and water. This animal experiments were approved by the Institutional Animal Care and Use Committee of the College of Medicine, National Taiwan University, and conducted in compliance with the guidelines outlined in the Guide for the Care and Use of Laboratory Animals.
II. Animal treatments: 2×10⁶ B16-F10 cells were subcutaneously injected into the right abdomen of mice to establish animal model with xenograft tumors. Once tumor volumes reached approximately 200 mm³, mice were randomly assigned to the three groups (n=5 per group), and subjected to medical treatment as shown in Table 2. All mice were sacrificed on Day 15. Further, the calculation formula of the tumor volume was: tumor volume=0.5×(tumor length)²×(tumor width).
(2) Analysis:
I. mice weights: The mice of each group were weighed every 5 days from Day 0.
II. Spleen weight analysis: After the mice of each group were sacrificed, the spleens thereof were taken and weighed.
III. Tumor Analysis: After the mice of each group were sacrificed, the tumors thereof were taken for appearance inspection, weight measurement, volume calculation and tumor sections for staining. The staining was carried out by the kit of Click-iT Plus TUNEL Assay Kits for In Situ Apoptosis Detection (purchased from Invitrogen) according to the manual instructions. DAPI nuclear staining was carried out for verification. Finally, the staining results were observed with a confocal microscope.
(3) Results: According to FIG. 16, the mice body weight of all groups showed no significant change during the experimental treatment, which indicated that GA-SeNPs were non-toxic to mice and demonstrated good tolerance. According to FIG. 17, the mice showed strong immune responses to the exogenous xenograft tumors; wherein both the GA-SeNPs (6 injections) group and GA-SeNPs (9 injections) group showed a lighter spleen weight than the PBS group, as their tumors were inhibited. Further, although systemic immune system activation would also increase the spleen weight, FIG. 17 showed that the spleen weights of the three groups were not similar and the spleen weight was positively correlated with the tumor size, which indicated that when GA-SeNPs promoted immune responses, a non-systemic or local immune system was activated. According to FIG. 18 to FIG. 21, in comparison with the PBS group, both the GA-SeNPs (6 injections) group and GA-SeNPs (9 injections) group significantly reduced the volume and weight of tumor and induced tumor cell apoptosis. Therefore, GA-SeNPs indeed had anticancer efficacy. Further, the scale bar in FIG. 18 was 1 cm, and the scale bar in FIG. 21 was 500 microns.

To sum up, the gluconic acid-coated selenium nanoparticles of the present invention have the following advantages: First, the preparation method thereof is simple. Second, GA-SeNPs (1) demonstrate good drug tolerance; (2) repolarize macrophages from an M2 macrophage to an M1 macrophage; (3) promote the generation of reactive oxygen species and cancer cells apoptosis; (4) increase tumor necrosis factor-α secretion and reduce interleukin-10 secretion; and (5) inhibit cancer. Finally, the present invention can be administered to the lesion directly, which facilitates precision medicine.

What is claimed is:

1. A gluconic acid-coated selenium nanoparticle, comprising a selenium nanoparticle and gluconic acid, wherein the gluconic acid is coated on the selenium nanoparticle.

2. The gluconic acid-coated selenium nanoparticle as claimed in claim 1, wherein the selenium nanoparticle has an average diameter of 20 nm to 250 nm.

3. The gluconic acid-coated selenium nanoparticle as claimed in claim 1, wherein the gluconic acid-coated selenium nanoparticle has an average diameter of 25 nm to 300 nm, and the gluconic acid-coated selenium nanoparticle has a Zeta potential of −10 mV to −50 mV.

4. A pharmaceutical composition, comprising the gluconic acid-coated selenium nanoparticle as claimed in claim 1 and a pharmaceutically acceptable carrier.

5. The pharmaceutical composition as claimed in claim 4, wherein the gluconic acid-coated selenium nanoparticle is in a concentration of 0.01 μg/g to 0.2 μg/g.

6. The pharmaceutical composition as claimed in claim 4, further comprising granulocyte-macrophage colony-stimulating factor, lactate oxidase or a combination thereof.

7. The pharmaceutical composition as claimed in claim 6, wherein the granulocyte-macrophage colony-stimulating factor is in a concentration of 10 ng/g to 50 ng/g, and the lactate oxidase is in a concentration of 0.01 U/g to 0.05 U/g.

8. The pharmaceutical composition as claimed in claim 4, wherein the pharmaceutical composition is in an injection dosage form.

9. A method for macrophage polarization, comprising contacting the pharmaceutical composition as claimed in claim 4 with a macrophage.

10. The method as claimed in claim 9, wherein the macrophage polarization comprises repolarization from an M2 macrophage to an M1 macrophage.

11. A method for cancer treatment, comprising administering to a subject in need thereof an effective amount of the pharmaceutical composition as claimed in claim 4.

12. The method as claimed in claim 11, wherein the cancer comprises melanoma.

13. A method for immune response promotion, comprising administering to a subject in need thereof an effective amount of the pharmaceutical composition as claimed in claim 4.

14. The method as claimed in claim 13, wherein the immune response promotion comprises promoting reactive oxygen species generation, inducing apoptosis at lesion, increasing tumor necrosis factor-α secretion, reducing interleukin-10 secretion or a combination thereof.

15. A manufacturing method for the gluconic acid-coated selenium nanoparticle as claimed in claim 1, comprising:
a mixing step: mixing sodium selenite, glucose, polyvinylpyrrolidone and water to obtain a mixture;
a heating step: heating the mixture to obtain a dark reddish-brown solution; and
a cooling step: lowering a temperature of the dark reddish-brown solution to obtain the gluconic acid-coated selenium nanoparticle.
